(19) United States
(12) Patent Application Publication
(10) Pub. No.: US 2014/0154153 A1
(43) Pub. Date: Jun. 5, 2014

(54) Test Specimen Holding Device for Inspecting Sterilization or Cleaning Processes

(71) Applicants: Elisabeth Averdam, Frankfurt am Main (DE); Reiner Leukel, Frankfurt am Main (DE); Burkhard Weg, Frankfurt am Main (DE); Martin Zink, Frankfurt am Main (DE)

(72) Inventors: Elisabeth Averdam, Frankfurt am Main (DE); Reiner Leukel, Frankfurt am Main (DE); Burkhard Weg, Frankfurt am Main (DE); Martin Zink, Frankfurt am Main (DE)

(73) Assignee: SANOFI-AVENTIS DEUTSCHLAND GMBH, Frankfurt am Main (DE)

(21) Appl. No.: 14/123,939

(22) PCT Filed: Jun. 6, 2012
(86) PCT No.: PCT/EP2012/060636

§ 371 Date: Jan. 23, 2014

(30) Foreign Application Priority Data
Jun. 6, 2011 (EP) 11168768.7

(51) Int. Cl. A61L 2/28

(52) U.S. Cl. CPC A61L 2/28
USPC 422/561

(57) ABSTRACT

The present invention relates to a test specimen holding device for inspecting a sterilization- and/or cleaning process. The holding device comprises: a substantially spherically shaped body comprising at least one through opening for a cleaning- and/or sterilization agent to enter the body, at least one receptacle to receive and/or to keep at least one test specimen inside the body.

### CROSS REFERENCE TO RELATED APPLICATIONS

The present application is a U.S. National Phase Application pursuant to 35 U.S.C. §371 of International Application No. PCT/EP2012/060636 filed Jun. 6, 2012, which claims priority to European Patent Application No. 11168768.7 filed Jun. 6, 2011. The entire disclosure contents of these applications are herewith incorporated by reference into the present application.

### FIELD OF INVENTION

The present invention relates to the field of sterilization- and/or cleaning processes and in particular to inspection, surveillance and characterization of such processes especially in a medical or pharmaceutical environment.

### BACKGROUND

In the medical and pharmaceutical environment, a manifold of items have to fulfil pre-defined sterile conditions. There exist various cleaning and sterilization standards which have to be constantly controlled and monitored. For such monitoring or surveying of sterilization and cleaning processes, sterilization specimen holders are typically used having a body featuring an internal chamber region. Such specimen holders keeping a test specimen inside, may enter a sterilization or cleaning process in an identical or similar way as the genuine items that are to be cleaned or sterilized.

For instance, WO 2009/014929 A1 discloses such a sterilization challenge specimen holder comprising a cap sealable on a first end of a body defining an internal chamber region. There, another plug is positioned in a second end of the body having an outer wall with a spiral-formed groove therein being in communication with the internal chamber region. Said plug further has an opening therein for defining a single flow path for communication between the environs and the internal chamber via the spiral formed groove. The body of this specimen holder is rather elongated and of substantially cylindrical shape.

If for instance the sterilization or cleaning process to be monitored involves continuous transportation of the items to be sterilized, a cylindrical shape of a test specimen holder may come along with some drawbacks. First of all, in an automated sterilization process, an elongated or cylindrically shaped specimen holder may get jammed or stuck in an automated sterilization- or cleaning plant. Additionally, such elongated holders—due to their size—are likely to become damaged when passing through an automated sterilization or cleaning process.

Additionally, a rather large and elongated specimen holder may disturb transport of the items to be sterilized in a cleaning or sterilization plant. Moreover, when the body of the specimen holder comprises a rather closed structure, exchange of sterilization or cleaning agents between the environment and the inner volume of the specimen holder is rather limited.

It is therefore an object of the present invention to provide an improved specimen holding device for inspecting and/or monitoring sterilization and/or cleaning processes. The device should be less prone to mechanical damage. It should be robust and should be designed for multiple use. It is a further object of the invention to provide a specimen holding device that has a reduced or even negligible impact on a transportation process of a cleaning and/or sterilization plant.

### SUMMARY

The present invention provides a test specimen holding device for inspecting and/or monitoring a sterilization and/or cleaning process. The test specimen holding device has a substantially spherically shaped body comprising at least one through opening for a cleaning and/or sterilization agent or medium to enter the body. Furthermore, the holding device comprises at least one receptacle to receive and/or to keep a test specimen inside the body.

By providing a substantially spherically shaped body, smooth and trouble-free transport of test specimen holding device in a sterilization or cleaning process can be attained. The spherical outer shape of the specimen holding device may therefore reduce the tendency of such devices to get stuck or jammed in a sterilization or cleaning process. Moreover, by providing a substantially spherical shaped body, the overall or outer size of the test specimen holder can be reduced or optimized in terms of providing a maximum inner volume. The spherically shaped body inherently provides a compact shape of the specimen holding device and may therefore better adapt to the shape and dimensions of items to become cleaned or sterilized by the cleaning or sterilization process.

The substantially spherically shaped body may be circular symmetric but may also comprise an ellipsoidal shape. Generally, the outer shape and dimensions of the test specimen holding device may be adapted and modified according to the respective cleaning or sterilization process to be analyzed. Independent and irrespective of the outer shape and structure of the test specimen holding device, its body is designed without edges or edge-free. Edges or other protruding portions of the holding structure could otherwise support a canting of the holding device in a cleaning or sterilization plant.

According to a preferred embodiment, the body comprises a first shell structure and a second shell structure. The two shell structures comprise mutually corresponding fastening means for releasably interconnecting first and second shell structures in order to form the spherically shaped body. Preferably, the first and second shell structure each comprise a half shell that can be mutually interconnected and interlocked to provide a spherical body having a receptacle or a receiving space for a test specimen to become placed inside.

According to a further embodiment, the fastening means of the first shell structure comprises a threaded socket pointing towards the second shell structure. The threaded socket is therefore adapted to threadedly engage with a threaded rim of the second shell structure. By having two shell structures that can be mutually interconnected and fastened by mutually corresponding threaded sections, a substantially spherically shaped body can be attained, wherein access to the inner volume of the body can be provided by simply unscrewing and dismantling first and second shell structures, respectively.

According to a further aspect, first and second shell structures comprise mutually corresponding circumferential contact surfaces adjacently arranged to the fastening means of first and second shell structures. The contact surfaces typically extend radially inwardly from the outer surface of first and second shell structures towards mutually corresponding threaded portions of first and second shell structure, respectively. The mutually corresponding contact surfaces provide a stop feature when screwing together first and second shell structures.

According to another embodiment, first and/or second shell structures comprise a substantially spherically-, cylindrically- or rectangularly shaped receptacle adapted to receive a correspondingly shaped test specimen. The inner receptacle formed by first and/or second shell structures is adapted and designed to fit with the outer geometry of the test specimen to be positioned in said receptacle. Dimensions of test specimen and receptacle may be designed such, that the test specimen can be clamped inside the receptacle. Hence, by insertion of the test specimen inside the receptacle, the test specimen may become positionally fixed thereto.

According to a further aspect, the receptacle is particularly adapted to receive and to hold a data logging device. The test specimen holding device may further be equipped with at least one data logging device. By data logging device-supported survey, process parameters of the sterilization or cleaning process can be measured and stored according to a pre-defined time schedule during a sterilization and or cleaning process. Data logging devices are typically adapted to at least monitor temperature, pressure and/or humidity during a sterilization- or cleaning process the test specimen holding device and the data logging device are exposed to.

Moreover and according to another preferred embodiment, the holding device comprises a fixing disc to be fastened inside the body. The fixing disc is further adapted to fix the test specimen in the receptacle of the body. Typically, the fixing disc is to be releasably disposed in an intermediate section between first and second shell structures. The fixing disc may comprise an outer diameter substantially matching the inner diameter of an inner thread of the second shell structure. This way, the fixing disc can be positionally fixed inside the receptacle formed by first and second shell structure as soon as first and second shell structure are screwed together and/or when mutually corresponding contact surfaces of first and second shell structures mutually abut.

Size and geometry of the fixing disc may be individually adapted to the size of the receptacle and/or to the size and geometric shape of the test specimen to be placed inside the body.

In another aspect, the fixing disc divides the inner volume of the body in at least two compartments. Hence, the circumference and diameter of the fixing disc may match with the inner diameter of the receptacle or with the free inner volume of the body. By dividing the inner volume of the body into at least two compartments, the body is enabled to receive and to keep at least two different types of test specimens simultaneously.

For instance, while in one compartment a data logging device can be arranged and fixed as a first test specimen, the other compartment is free to receive different types of test specimens, such like bioindicators or similar indicating specimens comprising for instance bacterial endotoxins, which after passing through the sterilization and/or cleaning process may be further investigated in order to determine whether the sterilization or cleaning process fulfils pre-defined sterilization or cleaning requirements.

Moreover, the second compartment may also serve to receive original items to become sterilized and/or cleaned. For instance, the compartment or receptacle of the spherically shaped body may be adapted to receive and to hold numerous items like closure caps, pistons or similar components applicable to manufacture containers for liquid medicaments, such like carpules, ampoules, infusion bottles, or pre-filled syringes.

According to a further embodiment, the fixing disc comprises at least one through opening being adapted to receive a measuring tip of the test specimen. In particular, when the test specimen is designed as data logging device featuring a measuring tip, the data logging device may penetrate the through opening of the fixing disc and may abut with a socket portion with the fixing disc in order to keep the data logging device in position inside the receptacle of the spherically shaped body. The through opening for receiving the measuring tip of the test specimen is typically located in a central area or in the middle of the fixing disc.

According to a further embodiment, the fixing disc is removably arranged inside the body and further comprises a multiplicity of through openings. Therefore, the fixing disc allows the cleaning and/or sterilization agent to flow through the fixing disc, and therefore supports exchange of the cleaning- or sterilization medium between the compartments separated by the fixing disc.

Moreover and according to another preferred embodiment, also the body itself and its first and second shell structures comprise a multiplicity of through openings arranged across the outer surface of the body in order to enable the cleaning and/or sterilization agent to flow therethrough. The spherically shaped body comprises a perforated structure so as to provide a reduced or generally low level of flow resistance for the cleaning- and/or sterilization medium. Said medium of either gaseous and/or liquid type may freely flow through the spherically shaped body and the at least one test specimen located therein.

The through openings arranged across the outer surface of the body may comprise a circular symmetric shape. They may feature holes extending in radial direction towards the center of the spherically shaped body. Moreover, the through openings or holes may be arranged in a regular manner across the outer surface of the body of the holding device.

According to another aspect, the body and/or at least one of its first and second shell structures comprises a thermoplastic material. The body and/or at least one of its shell structures may be manufactured by way of injection moulding. This way, a perforated structure as well as the mutually corresponding fastening means of first and/or second shell structures can be manufactured in a single manufacturing process. Especially by introducing a multiplicity of through openings in the body, the general weight and mass of the test specimen holding device can be advantageously reduced.

Also, by introducing numerous holes and through openings into the body, the spherically shaped structure becomes less susceptible to mechanical damage. The through openings in the body may thus provide a mechanical shock absorbing functionality and may prevent development of cracks in the body when exposed to mechanical impact. The thermoplastic material to be used for the body and/or at least one of its shell structures should be temperature resistant. It should be further inert to the cleaning- and/or sterilization agent or medium to be used in the respective cleaning- and/or sterilization process.

For instance, thermoplastic material such like polyether ether ketone (PEEK) provides sufficient temperature and chemical stability for cleaning and sterilization processes typically conducted in the medical or pharmaceutical environment.

It will be further apparent to those skilled in the art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, a preferred embodiment of the invention will be described in detail by making reference to the drawings in which:

FIG. 1 schematically illustrates a test specimen holding device in a perspective view in closed configuration,

FIG. 2 perspectively illustrates first and second shell structures of the holding device according to FIG. 1 in released and separate configuration and

FIG. 3 shows an exploded view of the holding device according to FIGS. 1 and 2 with a data logging device to be positioned and fixed therein.

### DETAILED DESCRIPTION

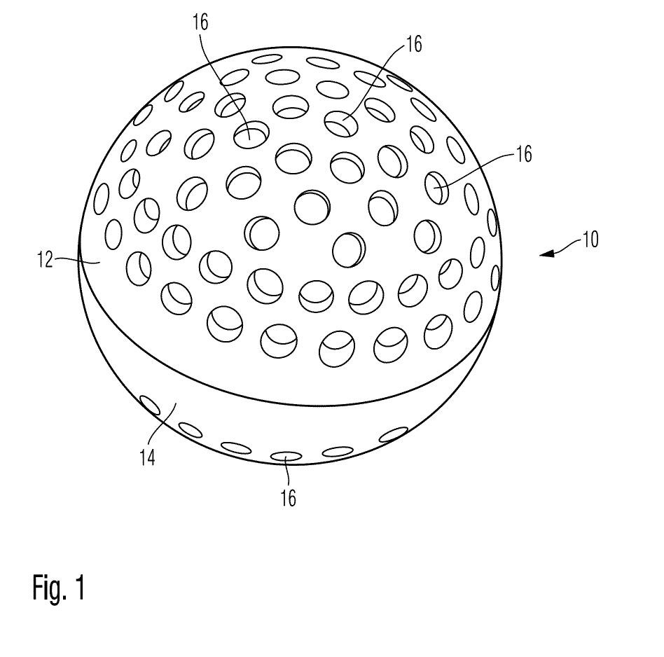

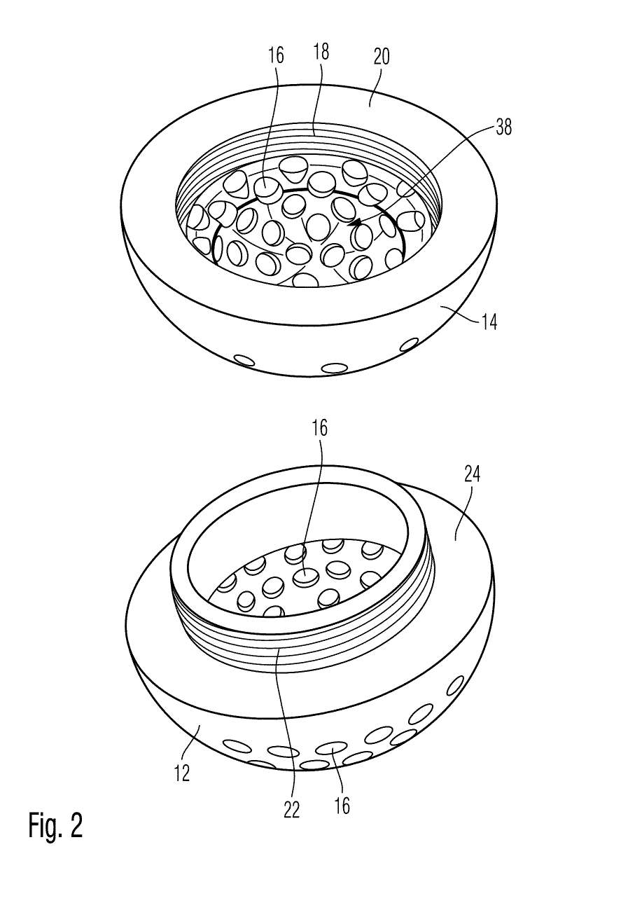

The test specimen holding device as depicted in closed or interlocked configuration in FIG. 1 as well as in released and dismantled configuration according to FIG. 2 comprises a substantially spherically shaped body 10, which is formed by screwing together two mutually corresponding shell structures 12, 14. Both shell structures 12, 14 comprise a half shell geometry and can be mutually connected and interlocked by way of a threaded engagement.

For this purpose, the shell structure 14 comprises an inner thread 18 adjacent to a circumferential and substantially plane shaped contact surface 20. Correspondingly, the other shell structure 12 comprises a socket having an outer thread 22. The threaded socket 22 protrudes in perpendicular direction to the plane of a contact surface 24 of the shell structure 12. Upon screwing together the two shell structures 12, 14 the threaded socket threadedly engages with the inner thread 18 until the contact surfaces 24, 20 of shell structures 12, 14 mutually abut. Then, the two half shell structures 12, 14 complement each other and form a spherically shaped body 12 as illustrated in FIG. 1.

The body 10 and its shell structures 12, 14 comprise a multiplicity of through openings allowing a cleaning or sterilization agent to enter the inner volume 38 of the body 10. The size of the through openings 16 is approximately in the range of the distance between adjacently located through openings 16. This way, the body 10 and/or its shell structures 12, 14 are rather perforated and feature a kind of lattice structure.

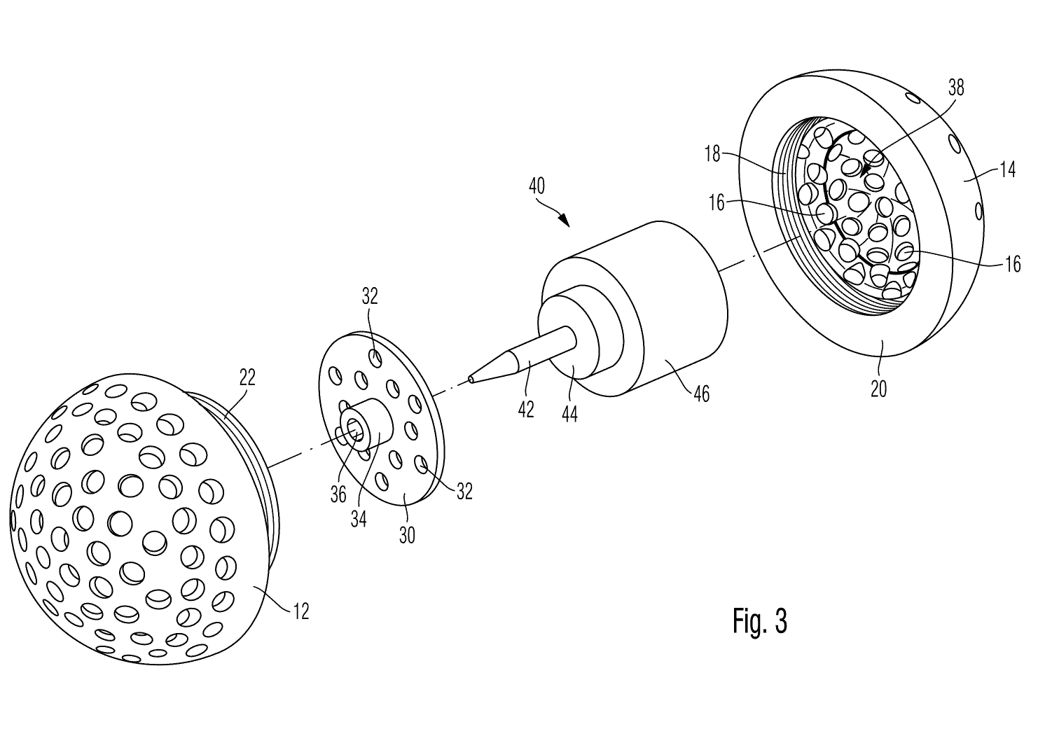

As illustrated for instance in FIGS. 2 and 3, the shell 14 comprises a somewhat cylindrically shaped receptacle 38 adapted to receive a cylindrically shaped body 46 of a test specimen 40. In the illustrated embodiment, the test specimen 40 comprises a data logging device having a body 46 and a stepped down socket portion 44, both of substantially cylindrical geometry. From the center of the socket portion 44 there extends a measuring tip 42.

During assembly, the data logging device 40 is positioned with its cylindrical body 46 in the correspondingly shaped receptacle 38 of the shell structure 14. Then, a fixing disc 30 having a central through opening 36 is put on top of the data logging device 40. The fixing disc 30 comprises a distance socket 34 around its central through opening 36 which is penetrated by the measuring tip 42 of the data logging device 40. After positioning of the fixing disc 30, some additional test items or specimens like bioindicators can be placed inside the shell structure 12 prior to a final assembly of first and second shell structures 12, 14.

The substantially even and flat shaped fixing disc 30 abuts with a lower side on the stepped down socket portion 44 when reaching a final assembly configuration. The circumference and diameter of the fixing disc 30 is chosen such that the fixing disc 30 fits into the inner thread 18 of the shell structure 14. The diameter of the fixing disc 30 may be almost as large as the outer diameter of the threaded socket 22 of the shell structure 12. This way, the fixing disc 30 can be immobilized and fixed inside the spherically shaped body 10 when the two half shells 12, 14 are screwed together.

By immobilizing the fixing disc 30 with respect to the shell structures 12, 14 and by placing or squeezing the data logging device 40 between the fixing disc 30 and a bottom section of the receptacle 38 of the shell structure 14, the data logging device 40 can be immobilized and secured with respect to the spherically shaped body 14. Since the fixing disc 30 is adapted to be fixed in a middle or central section of the body 10, the fixing disc 30 divides the inner volume of the body 10 into two compartments.

In the sketch of FIG. 3 the data logging device 40 occupies one compartment, whereas the other compartment on the opposite side of the fixing 30 disc is free to receive other types of test specimens, such like bioindicators, which are not explicitly illustrated here. By way of the central through opening 36 of the fixing disc 30 the measuring tip 42 may extend into the compartment adjacent the compartment occupied by the body 46 of the data logging device 40. However, the size of the spherically shaped body 10 and its shell structures 12,14 is designed such, that a distal end of the measuring tip does not protrude radially outwardly from the spherical surface of the test specimen holding device.

In fact, the measuring tip 42 is arranged entirely in the inner volume 38 defined by the two shell structures 12, 14. Since the shell structures 12, 14, comprise a multiplicity of through openings 16, even the measuring tip 42 can be washed round by e.g. a sterilization or cleaning agent in a respective sterilization- or cleaning process.

Also the fixing disc 30 comprises numerous through openings 32 allowing the sterilization or cleaning agent to penetrate therethrough. In this way, the test specimen holding device features a rather low flow resistance which allows to keep the impact of the specimen holding device to the cleaning or sterilization process as small as possible. Moreover, the rather perforated structure of the spherically shaped body 10 is beneficial to reduce overall weight of the holding device as well as to enhance its mechanical shock absorbing properties.

1-13. (canceled)

14. Test specimen holding device for inspecting a sterilization- and/or cleaning process, the holding device comprising:
a substantially spherically shaped body comprising at least one through opening (16) for a cleaning- and/or sterilization agent to enter the body,
at least one receptacle to receive and/or to keep at least one test specimen inside the body, characterized by
a fixing disc to be fastened inside the body and being further adapted to fix the test specimen in the receptacle of the body, and wherein the fixing disc comprises at least one through opening adapted to receive a measuring tip of the test specimen.

15. The holding device according to claim 14, wherein the body comprises a first shell structure and a second shell structure, said first and second shell structures comprise mutually corresponding fastening means for releasably interconnecting first and second shell structures.

16. The holding device according to claim 15, wherein the fastening means of the first shell structure comprises a threaded socket adapted to threadedly engage with a threaded rim of the second shell structure.

17. The holding device according to claim 15, wherein first and second shell structures comprise mutually corresponding circumferential contact surfaces adjacently arranged to the fastening means of first and second shell structures.

18. The holding device according to claim 15, wherein the first and/or the second shell structure comprise a substantially spherically-, cylindrically- or rectangularly shaped receptacle adapted to receive a correspondingly shaped test specimen.

19. The holding device according to claim 14, wherein the receptacle is adapted to receive and to hold a data logging device.

20. The holding device according to claim 14, wherein the fixing disc divides the inner volume of the body in at least two compartments.

21. The holding device according to claim 4, wherein the body is adapted to receive and to keep at least two different types of test specimens simultaneously.

23. The holding device according to claim 15, wherein the fixing disc is removably arranged inside the body and further comprises a multiplicity of through openings.

22. The holding device according to claim 14, wherein the body comprises a multiplicity of through openings arranged across the outer surface of the body to enable the cleaning- and/or sterilization agent to flow therethrough.

23. The holding device according to claim 14, wherein the body and/or at least one of its shell structures comprises a thermoplastic material.
